(19) United States
(12) Patent Application Publication
(10) Pub. No.: US 2002/0077687 A1
(43) Pub. Date: Jun. 20, 2002

(54) Catheter assembly for treating ischemic tissue

(76) Inventor: Samuel S. Ahn, Los Angeles, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 09/814,533

(22) Filed: Mar. 21, 2001

Related U.S. Application Data
(62) Division of application No. 09/736,844, filed on Dec. 14, 2000.

(51) Int. Cl. A61N 1/00; A61N 1/04; A61N 1/05; A61N 1/06

(52) U.S. Cl. 607/120; 604/164.13; 600/7

(57) ABSTRACT

The present invention provides for a catheter assembly for implanting cellular pellets into diseased or damaged heart muscle tissue. A guiding catheter is accurately positioned within either the left or right ventricle by means of an anchor wire so that a seeding catheter can distribute a pattern of cellular pellets into the diseased heart muscle tissue to stimulate heart muscle growth or angiogenesis.

### BACKGROUND OF THE INVENTION

Coronary heart disease is one of the leading causes of death in the United States. Heart attacks or myocardial infarctions caused by coronary heart disease can cause immediate death or can cause significant morbidity rates due to irreversible damage to the heart, such as scarring of the myocardial tissue.

Following a myocardial infarction there is always a certain time period of non-perfusion during which ischemia may develop. This is especially true during the patient transport to the hospital and until occluded vessels can be reopened by percutaneous transluminal coronary angioplasty (PTCA) or thrombolytic agents, for example. Thrombolytic agents, administered either intravenously or directly into the coronary arteries, work by dissolving the occluding thrombus and thereby reestablishing blood flow. When thrombolytic agents are administered properly, they can be expected to restore blood flow relatively quickly in cases of minor myocardial infarctions. However, in cases of massive myocardial infarctions, or in cases of delayed administration, the efficacy of the agents can be drastically reduced.

In situations where heart muscle damage has occurred due to myocardial infarctions or coronary heart disease, there have been attempts at improving perfusion in the damaged heart muscle and at repairing the heart muscle damage.

Some of the treatments have included attempts at growing microvessels through angiogenesis techniques. These techniques have experienced some significant drawbacks. The vessels that have been grown by these techniques have generally been too small in diameter and have provided little perfusion to the distant areas of the heart muscle, where perfusion is most needed. Also, most previous attempts such as U.S. Pat. No. 5,941,868 issued to Kaplan et al., involved injecting growth factors into the bloodstream in the target area which resulted in limited uptake into the heart muscle. These designs were at best only able to relieve symptoms of angina, but provided no improvement of cardiac function and were not able to convert dead muscle area into working muscle.

Some of the treatments for revascularizing the myocardium have involved the creation of channels within the myocardium for providing oxygenated blood to myocardial cells without requiring coronary circulation.

U.S. Pat. No. 5,878,751 issued to Hussein et al., discloses stent and needle means for creating and maintaining a patent lumen in the diseased myocardium. The stent is carried into the myocardium through the heart wall on the outside of a needle and then the needle is withdrawn through the center of the stent.

U.S. Pat. No. 5,972,013 issued to Schmidt discloses a pericardial access device having a penetrating body axially mobile with the lumen of a guide tube. The guide tube includes a deflecting mechanism for deflecting the distal end of the penetrating body. In use, a patient's pericardium is contacted with the distal end of the guide tube and suction is applied to form a pericardial bleb. The penetrating body is axially mobilized distally within the lumen of the guide tube until the deflecting mechanism deflects the penetrating body to cause the penetrating end of the penetrating body to enter the bleb of the pericardial tissue at an angle oblique to the longitudinal axis of the guide tube. These prior art devices do not disclose opening the percardium to gain access to the heart tissue.

Some of the problems associated with prior art devices include the ability of the physician to control the position of a catheter once it is inside the heart, and to identify ischemic tissue versus healthy tissue. Generally, if access to the interior of the heart is achieved by first advancing a catheter through the femoral artery, through the aorta and past the aortic arch, the length of the catheter may be anywhere from 135 to 165 cm. With such a long catheter, numerous factors are introduced which are difficult to control, including the pushability of the catheter, its torqueability, flexibility, and typically its trackability over a guide wire. Moreover, once the catheter is advanced into the heart, and typically into the left ventricle, it is difficult to determine ischemic tissue versus healthy tissue. What is needed, and has heretofore been unavailable, is a catheter assembly for treating ischemic tissue which resolves the problems associated with the prior art devices.

Accordingly, what is needed is an implant of cellular and pharmacological materials with the ability to regenerate heart muscle damaged from cardiac arrest or coronary disease, to improve cardiac function, and to stimulate angiogenesis. Also what is needed is a catheter-based deployment system for introducing the cellular implant into the heart wall in a minimally invasive procedure and to ensure that a predetermined delivery pattern can be achieved to ensure maximum benefit.

### SUMMARY OF THE INVENTION

The present invention meets the above-described need by providing myocardial cellular pellets and a system for deploying the pellets directly into the heart muscle in a pattern or circular array designed to repair the damaged tissue. Reference to cellular pellets herein is not meant to be limiting and includes cell suspension (i.e., cells suspended in a viscous liquid) or other combinations of cells mixed in a nutrient or therapeutic compound or angiogenic factors such as solutions of protein chemicals or other pharmacological agents or gene therapy.

The present invention generally provides for a cellular pellet comprised of a combination of cellular materials and/or pharmacological materials that are implanted directly into the heart muscle through the use of a catheter-based deployment system. The present invention generally provides for a catheter assembly for delivering a pattern of cellular pellets which are comprised of a combination of cellular materials and/or pharmacological materials that are implanted directly into heart muscle. The catheter assembly is configured to deliver a pattern of cellular pellets in heart muscle that has been damaged, typically by the lack of blood flow through the coronary arteries.

The catheter assembly for implanting a pattern of cellular pellets into the heart muscle includes a guide catheter having an elongated tubular body and a proximal and distal end. The elongated tubular body has at least two lumens, one lumen for carrying an anchor wire, and a second lumen for carrying a seeding catheter for delivering the cellular pellets. An elongated anchor wire is sized for slidable movement through the anchor wire lumen and it has a needle-shaped end or optionally may have a barb or hook at its distal end for attachment to the heart muscle wall. An elongated seeding catheter is sized for slidable movement through the seeding catheter lumen and is generally known in the art. A distal end of the guide catheter is positioned within the left ventricle of the heart so that the distal end of the anchor wire can penetrate and attach to the heart muscle wall or go through the ventrical wall and be pulled from outside the heart. Thereafter, the seeding catheter is advanced into the left ventricle and a series of cellular pellets are implanted by the seeding catheter in a pattern around the anchor wire. Preferably, the seeding catheter should be rotatable within the seeding catheter lumen and may have an articulating distal tip that can be angulated while the seeding catheter is in the ventricle to implant the desired pattern of cellular pellets around the anchor wire. The guide catheter assembly may be configured with an anchor wire lumen that has an exit port proximal of the distal end of the catheter, but closer to a distal end, in the so-called rapid-exchange or monorail configuration.

In one embodiment of the invention, the anchor wire is advanced distally out of the anchor wire lumen and into the heart muscle wall as previously described. Thereafter, the anchor wire is further advanced distally so that it goes through the endocardium, the myocardium, and the epicardium so that it completely penetrates the wall of the heart. Thereafter, the distal end of the anchor wire is grasped with an appropriate grasping device which has been inserted through a cannula or endoscope so that the anchor wire can be pulled out of the patient's body where it can be secured so that it provides an anchor about which the seeding catheter can then inject cellular pellets or cell suspension into the ischemic tissue. In one embodiment, the seeding catheter can be advanced from outside the patient through the rib cage, for example, over the distal end of the anchor wire and through the cannula or endoscope and adjacent to the epicardium. The cellular pellets or cell suspension can then be implanted into the myocardium with the result that the cellular pellets or cell suspension is implanted in the myocardium using both the epicardio and the endocardio approach to the area of the ischemic tissue to repair the damaged heart muscle. After completion of the seeding process, the anchor wire can then be withdrawn from the patient either distally or proximally as desired by the physician.

In one embodiment of the invention, the aforementioned guide catheter assembly is advanced through the femoral artery by insertion into a guiding catheter having a distal tip that has been pre-positioned past the aortic arch to facilitate entry into the left ventricle.

In one method of implanting the cellular pellets or cell suspension, the physician inserts a first guiding catheter through the femoral artery, through the aorta and past the aortic arch so that the distal end of the catheter is positioned to facilitate entry into the left ventricle. A second guide catheter, having dual lumens is inserted into the first guide catheter. An anchor wire is positioned within an anchor wire lumen of the second guide catheter, and a seeding catheter is positioned in the seeding catheter lumen of the second guide catheter. The second guide catheter, with the anchor wire and the seeding catheter in their respective lumens, but not advanced out of the second catheter, is advanced through the first guide catheter and past the aortic valve and into the left ventricle of the heart. The second guide catheter distal tip is positioned within the left ventricle, for example, by using intravascular ultrasound (IVUS) or fluoroscopy and a series of radiopaque markers on the catheters, the anchor wire, and the seeding catheter. The anchor wire is advanced distally out of the second guide catheter and, since it is relatively stiff, is inserted into the heart muscle wall that has been previously damaged. The anchor wire stabilizes the second guide catheter assembly and provides a focal point for the seeding catheter to implant the cellular pellets. The seeding catheter is next advanced out of the second guide catheter so that its distal end repeatedly penetrates the heart muscle wall at various locations and implants cellular pellets at a number of positions around the anchor wire. The seeding catheter has an articulated end, controlled by a control wire extending outside the patient, so that the end of the catheter can be angulated to implant cells in a pattern around the anchor wire. After the cellular pellets have been implanted, the seeding catheter is withdrawn proximally into the second guide catheter and the anchor wire is also withdrawn into the second guide catheter. The second guide catheter assembly is withdrawn proximally through the first guide catheter and out of the patient. The first guide catheter may remain in the patient for subsequent procedures, such as viewing the implanted area with known devices such as intravascular ultrasound (IVUS), optical fibers and the like.

In an alternative method of implanting the cellular pellets, the procedure is the same as previously described, except for the advancement and location of the anchor wire. The anchor wire is advanced distally out of the second guide catheter so that the anchor wire penetrates the myocardium and travels along the myocardium in a somewhat looping manner to better stabilize the anchor wire and hence the guiding catheter for implanting the pattern or array of cellular pellets.

In an alternative method of implanting the cellular pellets, the guide catheter is advanced into the inferior vena cava and into the right atrium where it then perforates the atrial wall into the left atrium and on into the left ventricle. At this point, the method of implanting cellular pellets or a cellular suspension is the same as described as when the guide catheter is advanced from the aortic arch through the left atrium and into the left ventricle. In yet another alternative method of implanting the cellular pellets, the guide catheter is advanced into the inferior vena cava and into the right atrium where it is further advanced into the right ventricle. The catheter then perforates the ventricular septum and is advanced into the left ventricle where the method of implanting is the same as previously described.

In a further alternative embodiment, the anchor wire penetrates the myocardium and is retrieved by a scissor-like clamp or grasper positioned on the outside of the heart through a cannula by known means. The grasper is used to grasp the distal end of the anchor wire and thread it back into the myocardium where the anchor wire stabilizes the second guide catheter for implanting the cellular pellets.

While the invention and its method of use as described herein has focused on repairing heart tissue, the device is not limited for use to repair the heart. For example, the catheter assembly can be used to repair any body tissue in any hollow organ such as the kidneys, liver, brain, gastrointestinal tract, esophagus, or pancreas.

Further features and description of the invention will appear from the following detailed description and in conjunction with the accompanying drawings.

### BRIEF DESCRIPTION OF THE DRAWINGS

The invention is illustrated in the drawings in which like reference characters designate the same or similar parts throughout the figures of which:

FIG. 1 is a perspective view of a heart having a damaged area in the myocardium.

FIG. 2 is a perspective view of the heart undergoing the procedure of the present invention.

FIG. 2A is an enlarged partial view of FIG. 2 depicting the portion of the heart requiring repair.

FIG. 3 is a perspective view of the heart after the damaged muscle tissue has been regenerated according to the method of the present invention.

FIG. 4 is a perspective view of a guiding catheter assembly for use with the present invention.

FIG. 5 is a side elevational view, partially in section, of one embodiment of a dual lumen catheter according to the present invention.

FIG. 6 is a cross-sectional view taken along the line 6-6 of FIG. 5.

FIG. 7 is a cross-sectional view taken along the line 7-7 of FIG. 5.

FIG. 8 is an enlarged partial cross-sectional view of a portion of the heart depicting the catheter of the present invention in the left ventricle.

FIG. 9 is an enlarged plan view of the anchor wire positioned in the myocardium and the seeding catheter implanting cellular pellets in heart tissue.

FIG. 10 is a plan view showing the distal end of the catheter of the invention having an adjustable, articulating tip portion.

FIG. 11 is an enlarged plan view of the anchor wire positioned in the myocardium and the seeding catheter implanting cellular pellets in heart tissue.

FIG. 12 is an enlarged plan view of the anchor wire piercing the myocardium and out the epicardium and a clamping tool threading the anchor wire back into the myocardium to stabilize the catheter assembly.

FIG. 13 is an enlarged partial cross-sectional view depicting placement of an endoscope through the rib cage area and having a grasper positioned adjacent the heart muscle wall.

FIG. 14 is a partial cross-sectional view of the heart and of the endoscope positioned adjacent to the epicardium so that the seeding catheter can be advanced into the myocardium.

FIG. 15 is a partial cross-sectional view of the heart depicting a second seeding catheter advanced into the myocardium for implanting a cell suspension or cellular pellet.

### DETAILED DESCRIPTION OF THE PREFERRED EMBODIMENTS

The present invention provides for a dual lumen guide catheter assembly for use in implanting a cell suspension or cellular pellets into diseased or damaged heart muscle. The catheter assembly is configured for placement in the left ventricle so that the cellular pellets can be implanted from inside the heart to repair a damaged area, typically caused by the lack of blood flow due to coronary artery disease.

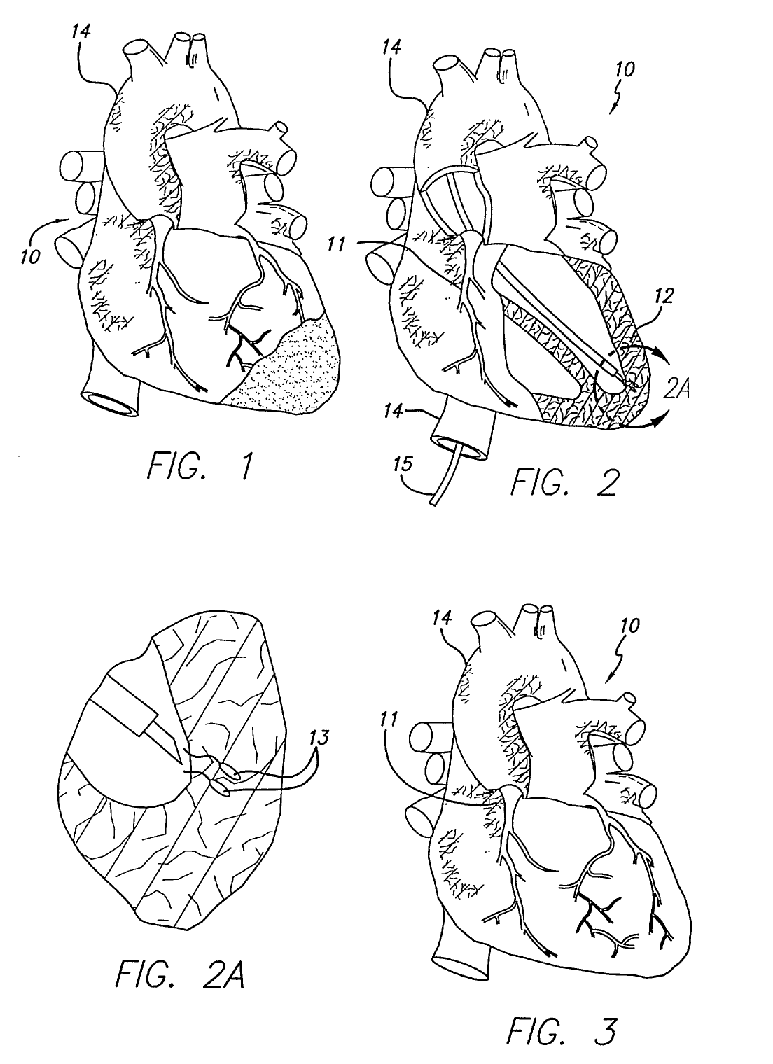

In FIG. 2, a heart 10 is shown having a damaged portion 12 where inadequate perfusion from the coronary artery 11 has led to damage to the heart muscle that results in diminished cardiac function and resulting morbidity.

Turning to FIG. 2, the device and method of the present invention provides for catheter-based deployment of a cell suspension or a cellular pellet 13 having a combination of cellular and pharmacological materials for regenerating heart muscle damaged from cardiac arrest or coronary disease, improving cardiac function, and stimulating angiogenesis in the muscle wall. Catheter assembly 15 is shown introduced through the aorta 14 and traversing through the aortic arch 14 and into the heart 10. As shown the catheter assembly implants the cellular pellet directly into the heart muscle wall from the inside of the heart.

The cellular pellet 13 preferably comprises the following materials: differentiated embryonic stem cells (cardiomyocytes), myoblasts, fibroblast growth factors, a biopsy of skeletal bone tissues, bone marrow tissue mixed with a biopsy of healthy heart muscle, nitric oxide synthase gene, pyruvate, catecholimine stimulating drugs, and fibrin glue. In one composition by volume is 60-90% (preferably 80%) differentiated embryonic stem cells, 5-20% (preferably 10%) growth factors, 1-10% (preferably 5%) nitric oxide synthase, 1-10% (preferably 2%) pyruvate, 1-10% (preferably 2%) fibrin glue and 1-10% (preferably 1%) catecholimine stimulating drugs.

The differentiated embryonic stem cells are cellular building blocks that have the ability to form new “beating” heart muscle cells that can proliferate and crowd out dead cells. The embryonic stem cells can be derived from donated human eggs for infertility clinics or from porcine eggs or from bone marrow tissue.

The growth factor is a protein that prompts the growth of new blood vessels. The protein, known as basic fibroblast growth factor or bFGF is a member of a class of drugs known as angiogenesis agents. Angiogenic agents include a variety of known growth factors such as fibroblast growth factors (FGF's), particularly including basic FGF (bFGF) and acidic FGF (aFGF); epidermal growth factor (EGF); platelet-derived growth factor (PDGF); vascular endothelial growth factor (VEGF); and the like. Such agents can prompt the body to grow new blood vessels giving blood new routes around clogged vessels. Accordingly, in the present invention the growth factors are used to grow new blood vessels to feed the heart muscle. The proteins are derived from laboratory culturing from human donation.

The nitric oxide synthase gene stimulates dilation of blood vessels and repair of endothelial linings and other functions essential to keeping tissue healthy. The material is available from laboratory culturing from human tissue donation.

The above components are injected alone or are incorporated into a cylindrical pressed tissue mass having dimensions of approximately 1 mm diameter by 3 mm length.

Alternative embodiments of the present invention can include different combinations of cellular material and pharmacological materials such as the following:

Pellet Composition #2, mesenchymal stem cells differentiated to cardiomyocytes, vascular endothelium growth factors, and nitric oxide synthase.

Pellet Composition #3, same composition as Pellet #1 described above except for the addition of electrical stimulation electrodes connected to a pacemaker-like stimulator implanted in the chest.

Pellet Composition #4, embryonic stem cells differentiated to cardiomyocytes, FGF and VEGF growth factors, fetal endothelial cells, placenta, cord blood, nitric oxide synthase, and pyruvate.

Pellet Composition #5, skeletal muscle cells, fetal endothelial cells, fibroblast and vascular endothelium growth factors, placenta, cord blood, and nitric oxide synthase.

All of the above compositions for cellular pellets may also include catecholamines and other cardiac output stimulating drugs. Also, the above-mentioned pellets can be shielded with a protective cork screw cage implanted in the muscle wall around the cellular pellet. The stainless steel corkscrew cage protects the pellet, assists in the anchoring of the pellet, and stimulates angiogenesis by injury and thrombus formation core.

Also, aspirin and Aldacatone can be added to the pellets. As another alternative, controlled gene expression technology can be incorporated into the cellular pellet 22.

Turning to FIG. 3, the damaged area 12 of the myocardial wall has been repaired by the device and method of the present invention and the result is healthy muscle tissue capable of significantly increasing cardiac function.

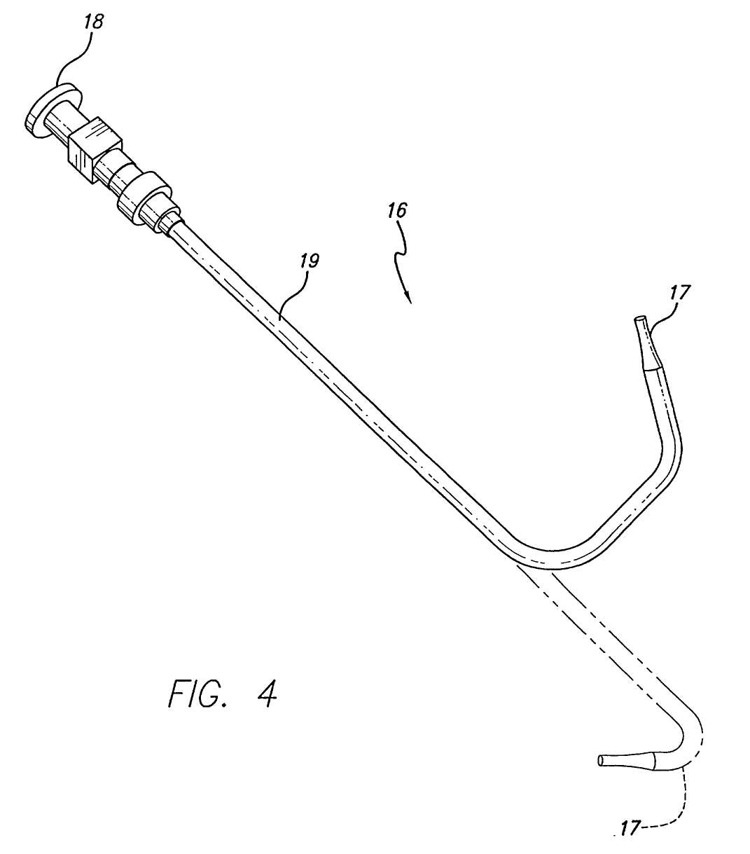

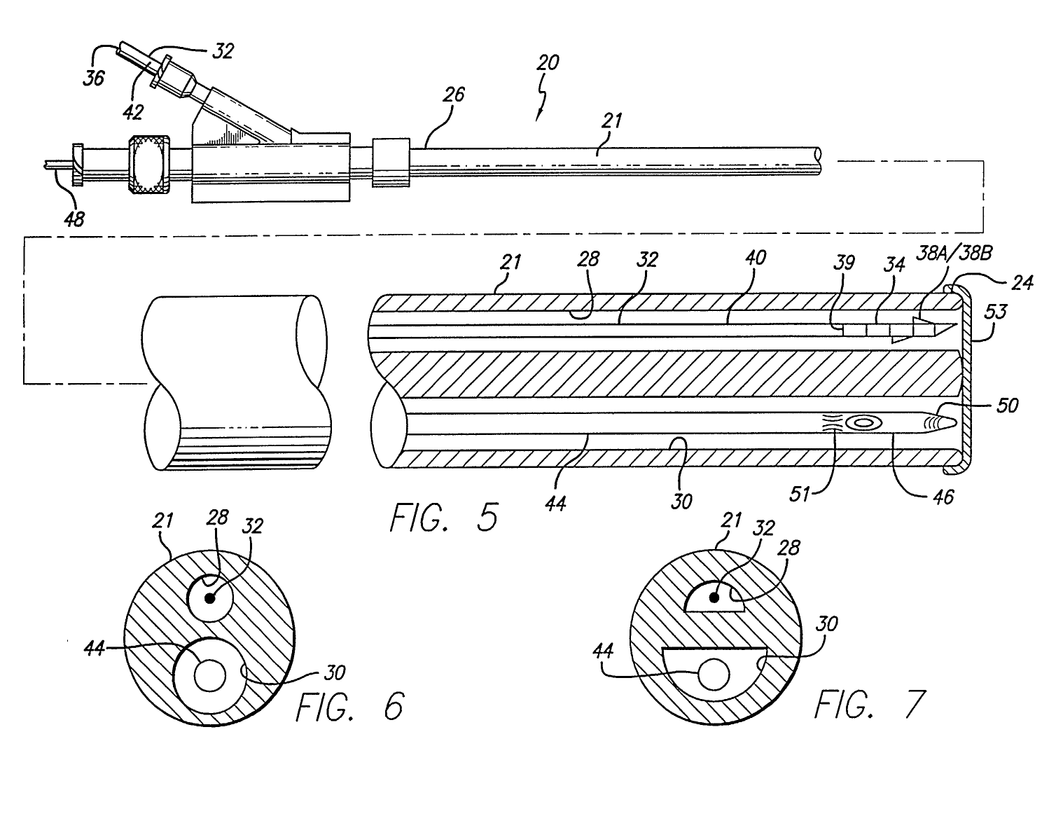

In keeping with the invention, as shown in FIGS. 4-7, a catheter assembly is provided which is configured to implant cellular pellets into diseased heart muscle. As shown in FIG. 4, a conventional first guiding catheter 16 has an elongated tubular member with a distal end 17 and a proximal end 18, with a through lumen 19 extending therethrough. The first guiding catheter is used in a conventional manner, typically through a femoral approach, or a brachial approach, to access the left ventricle. As shown in FIGS. 5-7, a second guide catheter 20 is formed of an elongated tubular member 21 having a distal end 24 and a proximal end 26. The second guide catheter is configured to have a dual lumen extending substantially from the distal end to the proximal end, each lumen having either a circular configuration as shown in FIG. 6, or a semicircular configuration as shown in FIG. 7. Other configurations for the dual lumens also are contemplated, and are a matter of design choice. An anchor wire lumen 28 extends from the distal end to the proximal end of the second guide catheter and is configured to slidably receive an anchor wire 32. The anchor wire has a distal end 34 and a proximal end 36 where the proximal end extends out of the patient so that it can be maneuvered by the physician. The distal end of the anchor wire may have an attachment device, such as a barb 38 for penetrating and attaching to the heart muscle wall. The anchor wire may have a stiff section 40 near its distal end, and a more flexible section toward the proximal end. The stiffer distal section is necessary in order to penetrate the heart muscle wall when the anchor wire is advanced distally out of the second guide catheter. A seeding catheter lumen 30 extends from the distal end to the proximal end of the second guide catheter and is configured for slidably receiving a seeding delivery catheter 44. The seeding catheter 44 has a distal end 46 and a proximal end 48, and a distal tip which is used to eject cellular pellets 13 into heart muscle wall 52. The seeding catheter may have an adjustable, articulating distal tip 51 (FIG. 10) that can be maneuvered to create an angle between the distal end of the seeding catheter and the distal end of the second guide catheter in order to distribute a more uniform pattern of cellular pellets into the heart muscle wall. For example, the distal tip 51 is articulated so that the angle between the distal tip and the anchor wire can be varied and controlled. A control wire 55 extends from out of the patient and is attached to the distal tip so that as the physician pulls proximally on the control wire, the distal tip articulates and forms a different angle relative to the anchor wire. Any number of angles can be created ranging from about 3° to about 45°. An optional protective membrane 53 is attached to the distal end 24 of the second guide catheter in order to protect heart muscle and sensitive tissue when the second guide catheter is advanced into the patient's vascular system, and eventually into the left ventricle. Further, the protective membrane prevents blood flow through the anchor wire lumen and the seeding catheter lumen, yet is flexible enough to permit the anchor wire and the seeding catheter to easily penetrate the membrane when they are advanced distally out of the second guide catheter.

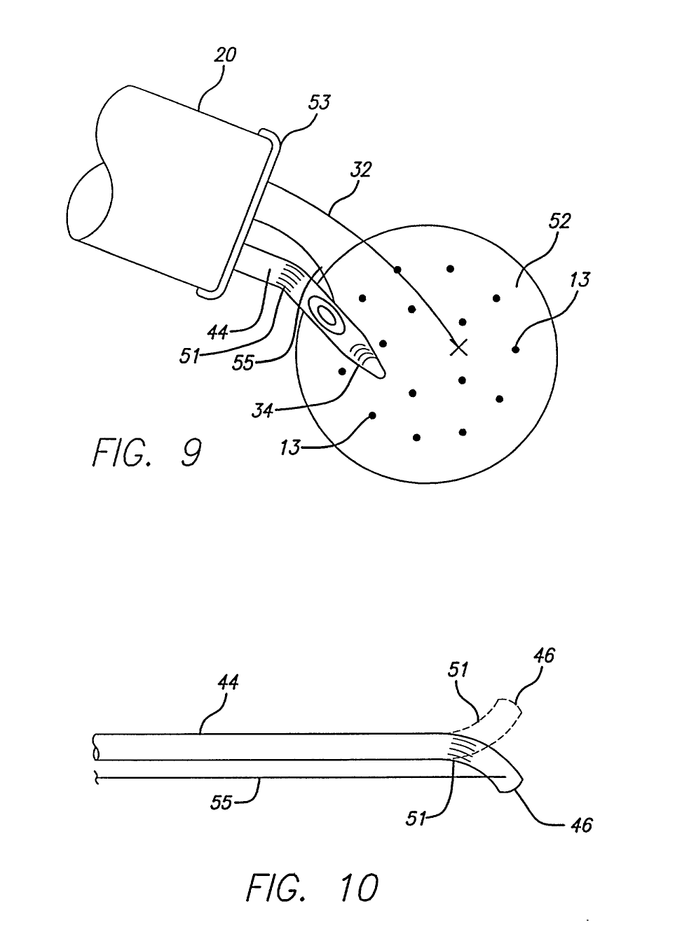

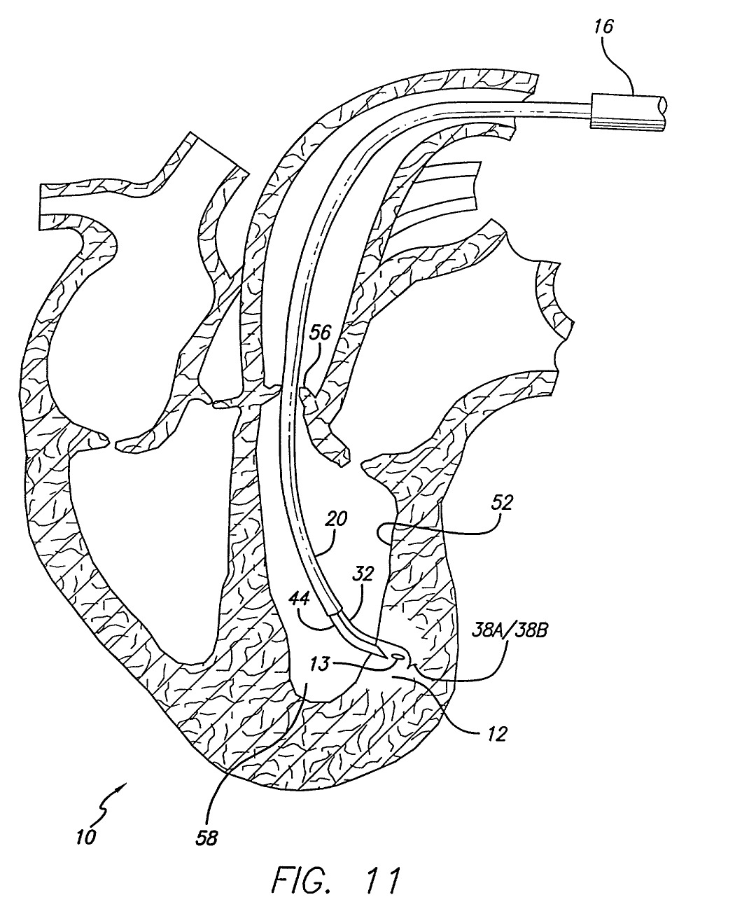

In keeping with the method of using the invention, as shown in FIGS. 5-10, after the first guide catheter 16 has been positioned in the aortic arch, the second guide catheter 20 is advanced through the through lumen 19 of the first guide catheter until the distal end 34 of the second guide catheter exits the distal end of the first guide catheter. The second guide catheter is further advanced distally past the aortic arch 54 and past the aortic valve 56 so that it is positioned within the left ventricle 58. The anchor wire 32 is next advanced distally out of the second guide catheter and it must penetrate the protective membrane 53 (optional) as it is advanced. The stiffened distal section 40 of the anchor wire provides enough pushability and torqueability to allow the physician to manipulate the proximal end 36 of the anchor wire so that the needle end 38A or barb 38B (if used) penetrates the heart muscle wall in the area of the damaged portion 13. The anchor wire is intended to stabilize the second guide catheter so that it has relatively little movement at its distal end in the ventricle. The anchor wire preferably has a plurality of radiopaque markers 39 at its distal end so that the physcian can view the position of the distal end of the anchor wire as it is being inserted into the heart muscle wall. In one embodiment, the anchor wire penetrates the myocardium, but preferably it does not pierce the pericardium. Thus, the radiopaque markers can be positioned at a specific distance form the distal tip of the anchor wire, such as at 1 mm intervals, in order to assist the physician in determining the depth of penetration of the anchor wire. The seeding delivery catheter 44 is next advanced through the protective membrane and is further advanced distally to penetrate the heart muscle wall where a cellular pellet or cell suspension can be implanted. The seeding catheter is repeatedly withdrawn from the heart muscle wall and reinserted into the heart muscle wall at another location near the anchor wire to distribute a circular array or pattern of cellular pellets around the anchor wire. The anchor wire remains positioned in the heart muscle wall to first locate the area of the damaged portion 12 as well as provide an anchor and a focal point for the distribution of the cellular pellets. After the cellular pellets have been implanted, the seeding catheter is withdrawn proximally into the second guide catheter and the anchor wire is then withdrawn proximally into the seeding catheter. The seeding catheter is then withdrawn proximally out of the first guide catheter and removed from the patient. The first guide catheter may remain in the patient for further procedures, including using fiber optics or other visual means to view the implanted area.

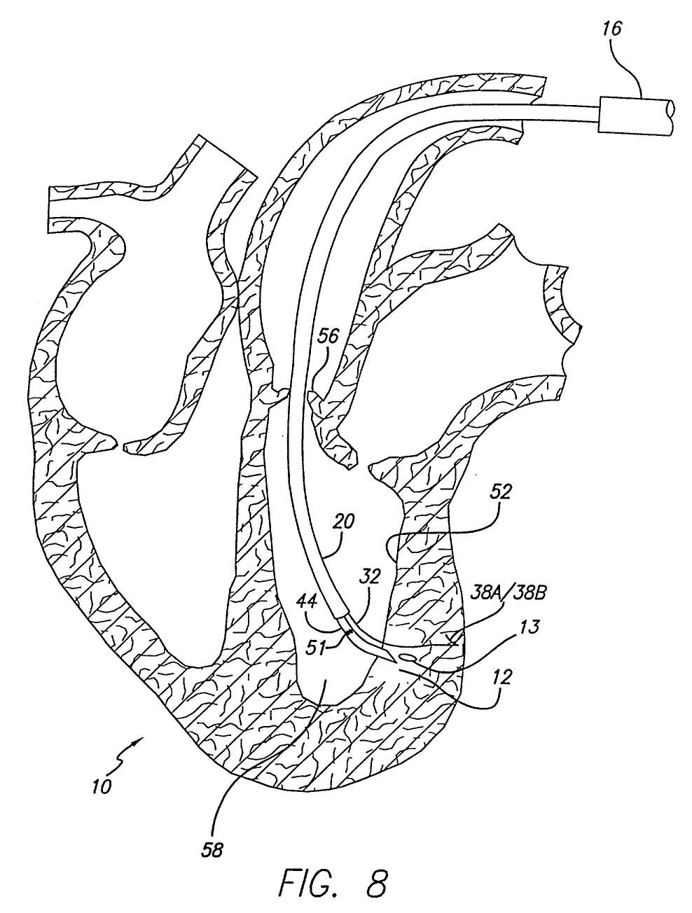

Alternatively, the second guide catheter 20 shown in FIGS. 8-10, can be advanced through the inferior vena cava, through the right atrium, through the right ventricle, and then pierce and advance through the septum into the left ventricle. At this point, the second guide catheter system is used to inject cellular cells as describe previously for the left ventricle approach (FIG. 8).

In an alternative method of using the invention, as shown in FIG. 11, the method is similar to that described with respect to FIGS. 5-10 except for the placement of the anchor wire. In this embodiment, the anchor wire is advanced distally out of the second guide catheter and into the heart muscle wall, and more particularly into the myocardium. It is looped within the myocardium as shown in FIG. 11 so that it provides a more stable base for the second guide catheter and the seeding delivery catheter 44.

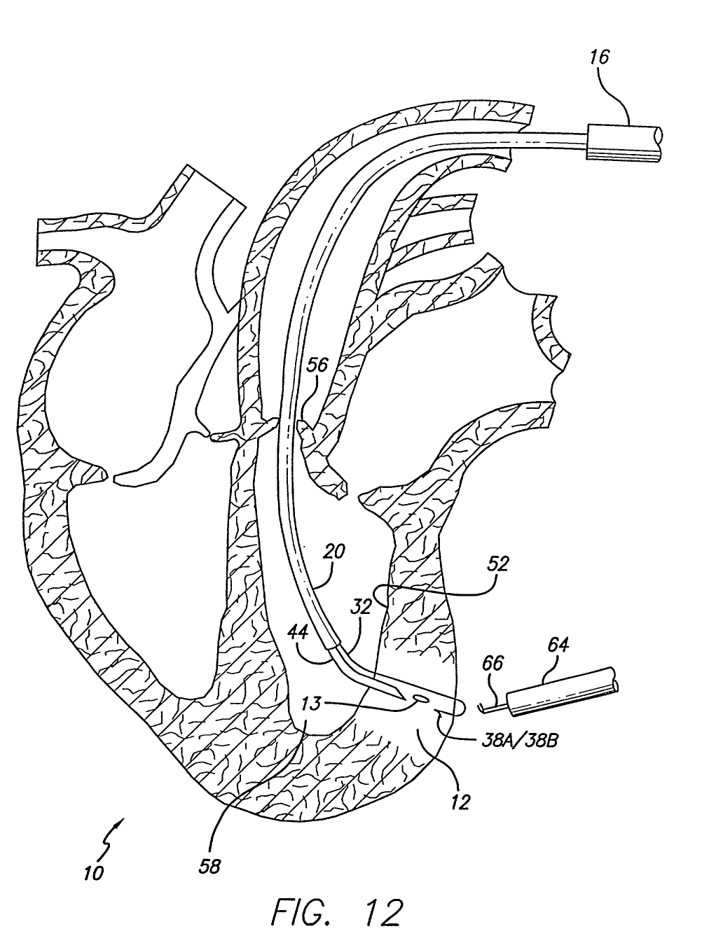

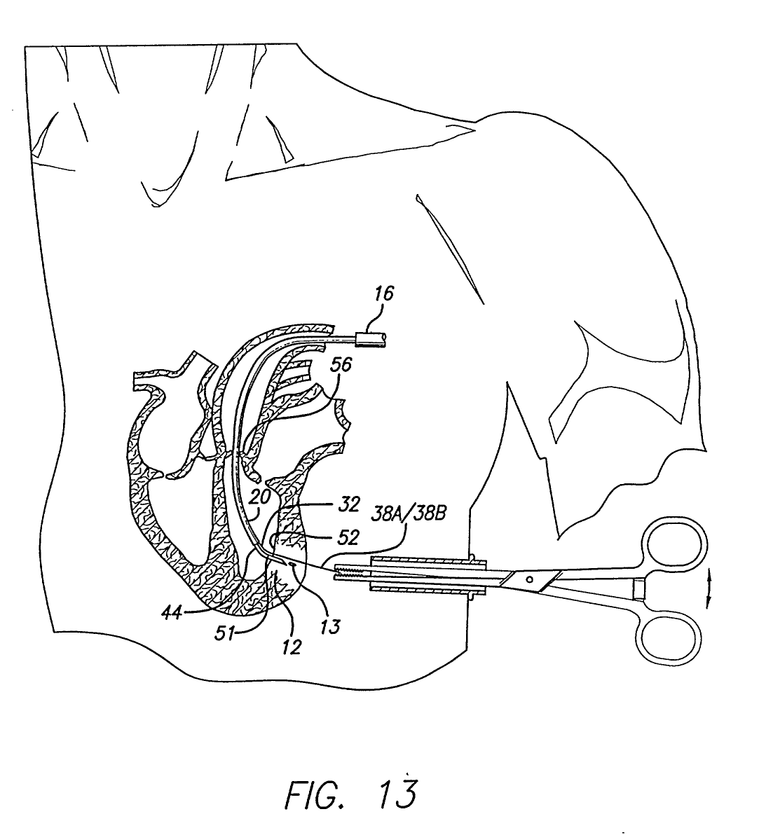

In a further alternative method of using the invention, as shown in FIG. 12, again the method is the same as that described for FIGS. 5-10 with the exception of the placement of the anchor wire. In this embodiment, the anchor wire is advanced distally out of the second guide catheter and through the myocardium so that it is outside the epicardium. A prepositioned cannula 64 is used to deliver a grasper or scissor clamp 66 for engagement with the anchor wire. The grasper is used to manipulate the anchor wire and push it back through the epicardium and into the myocardium so that the needle end 38A or the barb 38B on the anchor wire can engage the myocardium and form a looping configuration with respect to the heart muscle wall. In this configuration, the anchor wire provides a more stable assembly for the second guide catheter and the seeding delivery catheter 44. The method of implanting the cellular pellets is the same as previously described. When the seeding operation is completed, the distal end of the anchor wire can be cut with an appropriate tool inserted through the cannula 64. The very distal portion of the anchor wire would remain in the myocardium, and the remaining portion of the anchor wire would be withdrawn proximally through the guiding catheter and out of the patient.

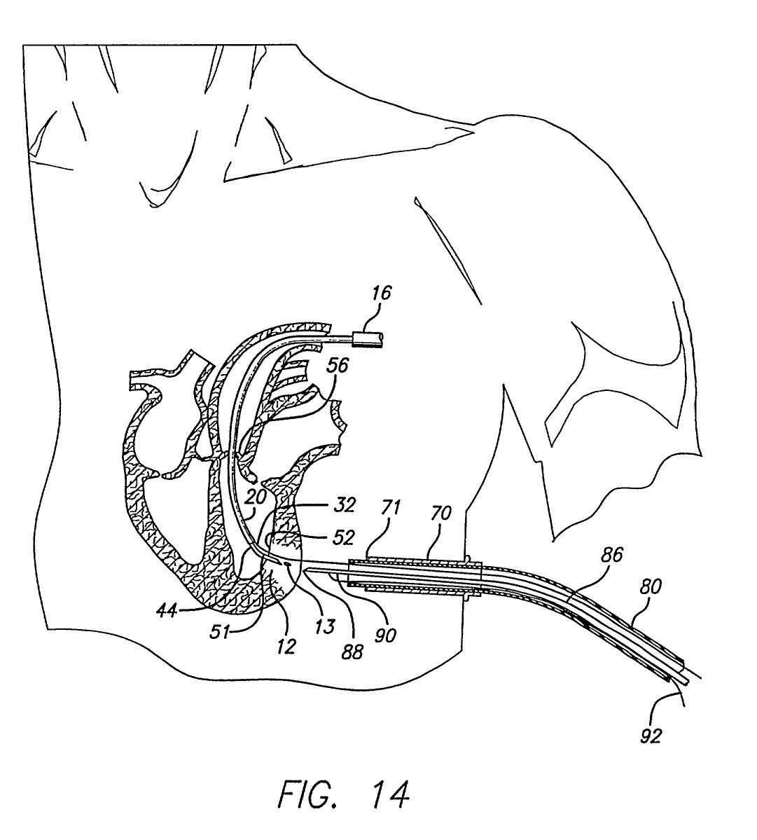

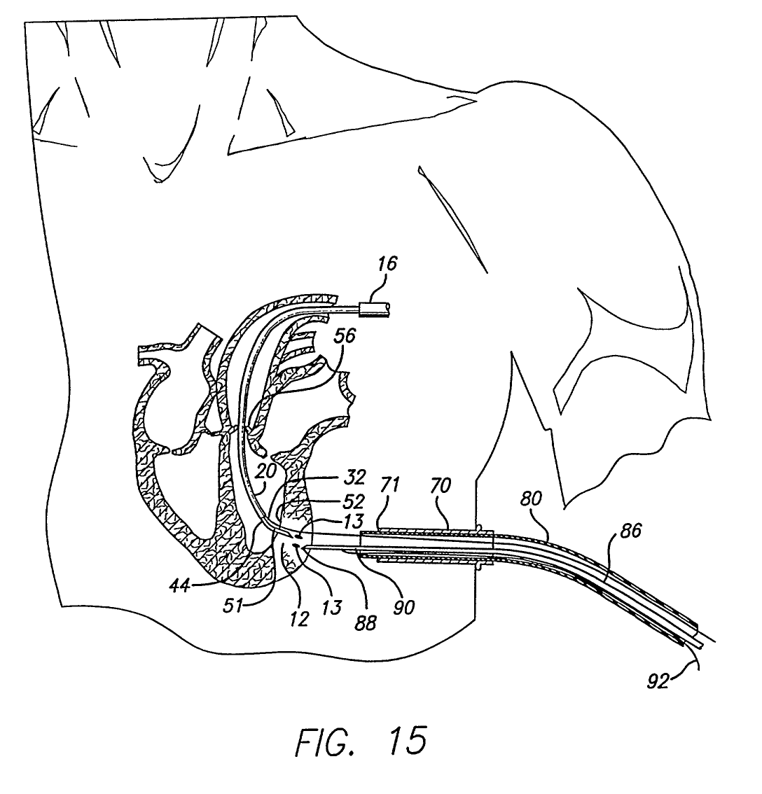

In a further alternative method of using the invention, it may be desirable to treat the damaged portion of the heart muscle on both sides of the heart muscle wall. In other words, the previously described treatment methods implant cellular pellets or cell suspension into the damaged tissue via the left ventricle. Thus, the myocardium is treated by the previously described method, and the myocardium often can be treated by access through the epicardium. In keeping with the invention, and shown in FIGS. 13-15, an endoscope 70, or similar device having at least two lumens, is inserted through the rib cage so that the distal end 71 of the endoscope is near the epicardium of the heart muscle wall. Placement of the endoscope is well known and a routine operation. A grasper 72, also well known, is advanced through the endoscope so that it is adjacent the epicardium. The anchor wire needle end 38 is advanced through the endocardium, through the myocardium and the epicardium so that it exits the heart muscle wall and is then grasped by the grasper 72 so that it can be pulled proximally through the endoscope. The anchor wire 32 is pulled through the endoscope proximally until it is out of the patient. Thereafter, a third guide catheter 80 is back loaded onto the anchor wire distal end 34 and advanced distally through the endoscope so that a distal end 82 of the third guide catheter is immediately adjacent the epicardium. Importantly, the third guide catheter travels along the anchor wire which is firmly fixed and extends through the heart muscle wall. A second delivery catheter 86 or seeding catheter is then advanced through the endoscope so that the distal end 88 of the second delivery catheter can be advanced into the myocardium for the purpose of implanting cellular pellets or cell suspension. As described previously, the second delivery catheter also has an articulating distal end 90, which can be articulated through use of control wire 92, to repeat the implanting procedure in an array or pattern around the anchor wire. The second delivery catheter can approach the heart from the outside at any angle, inferior or anterior, and the articulating distal end ensures that a pattern of cellular cells is distributed in the heart tissue around the anchor wire. After the second delivery catheter implants the cellular pellets or cell suspension in a pattern around the anchor wire, which should be adjacent the cellular implant pattern previously placed on the myocardium adjacent the anchor wire, the second delivery catheter can be withdrawn proximally from the patient. Thereafter, the anchor wire can be removed from the patient either by moving it distally or proximally as desired. The endoscope 70 is removed from the patient and the first guide catheter 16 and second guide catheter 20 also are removed from the patient after the procedure is completed.

The first guiding catheter typically can be formed of a material which has a very low coefficient of friction as for example TFE Teflon (a tetrafluoroethylene polymer) having a coefficient of friction of approximately 0.02. The first guide catheter should have a through lumen having an internal diameter of approximately 8 or 9 French, or less. The length of the first guide catheter is well known in the art, and may be on the order of 150 cm, or a dimension to suit a particular application.

The second guiding catheter can be made of a polymer material as well, and also should have an extremely low coefficient of friction such as TFE Teflon. Other materials can be used to form either the first or second guide catheters including fluorinated ethylene-propylene resins (FEP), polyethylene terephalate (PET), Hytrel polyesters, aromatic polymers, or polyethereketone (PEEK). Other materials of manufacture include block co-polymers, particularly polyamide-polyester block co-polymers with a tensile strength of at least 6,000 psi and an elongation of at least 300%, and polyamide or nylon materials, such as NYLON 12, with a tensile strength of at least 15,000 psi. The outer diameter of the second guiding catheter is sized so that it can be slidably received into the through lumen of the first guiding catheter. Typical dimensions of the second catheter can include an overall length of about 135 to about 175 cm with a tubular member outer diameter of about 0.035 to about 0.45 inch (0.635-1.14 mm). The inner lumens, namely the anchor wire lumen and the seeding catheter lumen are sized to receive the anchor wire and the seeding catheter and can vary according to a particular application. Generally speaking, the diameter of the anchor wire would be on the order of 0.014 to 0.20 inch and the seeding catheter would have an outer diameter on the order of 3 to 10 French. While the dimensions stated herein are for illustration purposes, it is clear that the dimension can vary significantly to suit a specific application.

Visual marking means are provided for locating the relative positions of the first guiding catheter, the second guiding catheter, the anchor wire, and the seeding catheter. For example, a radiopaque marker can be placed at the distal end of the first guiding catheter. Similarly, a radiopaque marker can be placed on the distal end of the second guiding catheter so that the physician can view the distal end of the guiding catheter as it is advanced through the patient's vascular system and into the right or left ventricles. Likewise, the distal portion of the anchor wire can have radiopaque markers or a radiopaque coating to identify the end of the anchor wire so that it can be more precisely positioned into the heart muscle wall. The seeding catheter also has distal radiopaque markers to identify its distal end for insuring accurate placement of the seeding catheter into the heart muscle tissue, and hence a more precise pattern of implanting cellular pellets is achieved. Radiopaque markers typically are made from gold, silver, platinum, tantalum, or other high density metals.

When the physician first advances the second guide catheter into the left ventricle so that the distal end of the second guide catheter is adjacent the myocardium, often it is difficult to determine healthy tissue from ischemic tissue to assist the physician in determining where to position the distal end of the second guide catheter in the left ventricle, the anchor wire is equipped with electrodes 90 which are able to differentiate healthy tissue from ischemic tissue. When the distal end of the anchor wire penetrates the myocardium, an electrical signal is generated through the anchor wire and it is monitored outside the patient by the physician. As the heart beats, the muscle cells generate electrical signals which are picked up by the anchor wire electrode and which can differentiate between the electrical signals generated through healthy tissue and those generated through ischemic tissue. The process generally is called monoaction potential or MAP.

While the catheter assembly of the invention has been described in terms of certain presently preferred embodiments, it should be apparent that modifications and improvements can be made without departing from the scope of the invention.

It is to be understood that although the present invention has been described in connection with percutaneous procedures, it is also suitable for open chest cavity procedures where the interventionist or surgeon has detected heart muscle damage during the open procedure. Also, although the present invention has been described in connection with the myocardium, the cellular pellet and deployment system is not to be limited to use only with the heart muscle wall and may be applied to other organs of the body with suitable cellular compositions formulated for use in other areas such as the liver, kidneys, pancreas, esophagus, G.I. tract, G.U. tract, or the brain.

While the invention has been described in connection with certain preferred embodiments, it is not intended to limit the scope of the invention to the particular forms set forth, but, on the contrary, it is intended to cover such alternatives, modifications, and equivalents as may be included within the spirit and scope of the invention as defined by the appended claims.

What is claimed:

1. A catheter assembly for implanting a pattern of cellular pellets into body tissue, comprising:
a guide catheter having an elongated tubular body and a distal end, and a proximal end, and having an anchor wire lumen and a seeding catheter lumen extending therethrough;
an elongated anchor wire sized for slidable movement through the anchor wire lumen;
an elongated seeding catheter sized for slidable movement through the seeding catheter lumen;
whereby the guide catheter distal end is positioned adjacent the body tissue so that a distal end of the anchor wire can be attached to the body tissue and the seeding catheter can be advanced through the guide catheter so that cellular pellets can be implanted in the body tissue in a pattern around the attachment point of the anchor wire.

2. The catheter assembly of claim 1, wherein the seeding catheter is rotatable within the seeding catheter lumen.

3. The catheter assembly of claim 2, wherein the seeding catheter has an articulating distal end to enhance distribution of the cellular pellets in a seeding pattern around the anchor wire.

4. The catheter assembly of claim 3, wherein the seeding catheter angulated distal end is formed from a flexible material.

5. The catheter assembly of claim 1, wherein the seeding catheter has a torquing member at the proximal end thereof.

6. The catheter assembly of claim 1, wherein a fiber optic cable is associated with the seeding catheter to view the seeding distribution.

7. The catheter assembly of claim 1, wherein magnets resonance imaging is used to view the seeding catheter.

8. The catheter assembly of claim 1, wherein an ultrasound device is associated with the seeding catheter to view the seeding distribution.

9. The catheter assembly of claim 1, wherein an imaging device is associated with the seeding catheter to view the seeding distribution.

10. The catheter assembly of claim 1, wherein the anchor wire has a barb on a distal tip thereof.

11. The catheter assembly of claim 1, wherein the guide catheter has at least one radiopaque marker.

12. The catheter assembly of claim 1, wherein the seeding catheter has at least one radiopaque marker.

13. The catheter assembly of claim 1, wherein the anchor wire has at least one radiopaque marker.

14. The catheter assembly of claim 1, wherein the anchor wire is formed from a material that provides variable stiffness along the length of the anchor wire.

15. The catheter assembly of claim 14, wherein the anchor wire has a stiff proximal section, a relatively flexible central section, and a relatively stiff distal section.

16. The catheter assembly of claim 1, wherein the anchor wire lumen and the seeding catheter lumen are coaxial along the length of the elongated guide catheter.

17. The catheter assembly of claim 1, wherein the anchor wire lumen is substantially smaller than the seeding catheter lumen.

18. The catheter assembly for claim 1, in which the anchor wire lumen extends in a parallel relationship to the seeding catheter lumen throughout the length of the guide catheter.

19. The catheter assembly of claim 1, wherein the anchor wire lumen has a distal exit port that coincides with the distal end of the elongated guide catheter, and a proximal exit port that is positioned between the guide catheter distal end and proximal end.

20. The catheter assembly of claim 1, wherein the seeding catheter lumen has a distal exit port that coincides with the guide catheter distal end, and a proximal exit port that is positioned at a point between the guide catheter distal end and proximal end.

21. The catheter assembly of claim 1, wherein the catheter assembly is configured for use in any hollow organ including kidneys, liver, brain, gastrointestinal tract, esophagus, and pancreas.

22. A catheter assembly for implanting a pattern of cellular pellets into body tissue, comprising:
a first guide catheter having a proximal end and a distal end and a lumen extending therethrough;
a second elongated tubular body and a distal end, and a proximal end, and having an anchor wire lumen and a seeding catheter lumen extending therethrough;
the first guide catheter lumen being sized for slidably receiving the second guide catheter;
an elongated anchor wire sized for slidable movement through the anchor wire lumen;
an elongated seeding catheter sized for slidable movement through the seeding catheter lumen;
whereby the guide catheter distal end is positioned adjacent the body tissue so that a distal end of the anchor wire can be attached to the body tissue and the seeding catheter can be advanced through the guide catheter so that cellular pellets can be implanted in the body tissue in a pattern around the attachment point of the anchor wire.

23. The catheter assembly of claim 22, wherein the seeding catheter is rotatable within the seeding catheter lumen.

24. The catheter assembly of claim 23, wherein the seeding catheter has an angulated distal end to enhance distribution of the cellular pellets in a seeding pattern.

25. The catheter assembly of claim 24, wherein the seeding catheter angulated distal end is formed from a flexible material.

26. The catheter assembly of claim 22, wherein the seeding catheter has a torquing member at the proximal end thereof.

27. The catheter assembly of claim 22, wherein the seeding catheter includes an imaging device to view the seeding distribution.

28. The catheter assembly of claim 22, wherein magnets resonance imaging is used to view the seeding catheter.

29. The catheter assembly of claim 22, wherein an ultrasound device is associated with the seeding catheter to view the seeding distribution.

30. The catheter assembly of claim 22, wherein an imaging device is associated with the seeding catheter to view the seeding distribution.

31. The catheter assembly of claim 22, wherein the anchor wire has a barb on a distal tip thereof.

32. The catheter assembly of claim 22, wherein the second guide catheter has at least one radiopaque marker.

33. The catheter assembly of claim 22, wherein the seeding catheter has at least one radiopaque marker.

34. The catheter assembly of claim 22, wherein the anchor wire has at least one radiopaque marker.

35. The catheter assembly of claim 22, wherein the anchor wire is formed from a material that provides variable stiffness along the length of the anchor wire.

36. The catheter assembly of claim 35, wherein the anchor wire has a stiff proximal section, a relatively flexible central section, and a relatively stiff distal section.

37. The catheter assembly of claim 22, wherein the anchor wire lumen and the seeding catheter lumen are coaxial along the length of the elongated second guide catheter.

38. The catheter assembly of claim 22, wherein the anchor wire lumen is substantially smaller than the seeding catheter lumen.

39. The catheter assembly for claim 22, in which the anchor wire lumen extends in a parallel relationship to the seeding catheter lumen throughout the length of the second guide catheter.

40. The catheter assembly of claim 22, wherein the anchor wire lumen has a distal exit port that coincides with the distal end of the second guide catheter, and a proximal exit port that is positioned between the guide catheter distal end and proximal end.

41. The catheter assembly of claim 22, wherein the seeding catheter lumen has a distal exit port that coincides with the second guide catheter distal end, and a proximal exit port that is positioned at a point between the second guide catheter distal end and proximal end.

42. The catheter assembly of claim 22, wherein the catheter assembly is configured for use in any hollow organ including kidneys, liver, brain, gastrointestinal tract, esophagus, and pancreas.
